(19) United States
(12) Patent Application Publication
(10) Pub. No.: US 2023/0294086 A1
(43) Pub. Date: Sep. 21, 2023

(54) CAPILLARY-DRIVEN COLORIMETRIC ASSAY DEVICES

(71) Applicant: Colorado State University Research Foundation, Fort Collins, CO (US)

(72) Inventors: Charles S. Henry, Fort Collins, CO (US); Brian J. Geiss, Fort Collins, CO (US); David S. Dandy, Fort Collins, CO (US); Cody Carrell, Sun Prairie, WI (US); Jeremy Link, Fort Collins, CO (US); Isabelle Samper, Fort Collins, CO (US); Ana Sanchez-Cano, Fort Collins, CO (US); Ilhoon Jang, Seoul (KR); Zachary Call, Fort Collins, CO (US)

(21) Appl. No.: 18/016,753

(22) PCT Filed: Jul. 29, 2021
(86) PCT No.: PCT/US21/71050

§ 371 Date: Jan. 18, 2023

Related U.S. Application Data
(60) Provisional application No. 63/058,683, filed on Jul. 30, 2020.

(51) Int. Cl. B01L 3/00; G01N 33/543

(52) U.S. Cl. CPC B01L 3/5023; G01N 33/54388; B01L 3/50273; B01L 2200/16; B01L 2400/0406; B01L 2300/069; B01L 2300/088

(57) ABSTRACT

An assay device includes a colorimetric testing assembly including a detection area, a fluid inlet, and a microfluidic network including a first path extending to the detection area and a second path extending to the detection area. When a fluid (e.g., a buffer fluid or a combined buffer and sample solution) is provided to the fluid inlet, a first portion of the fluid rehydrates a first dried reagent (e.g., a dried enzyme label) disposed along the first path to produce a first rehydrated reagent and a second portion of the fluid rehydrates a second dried reagent (e.g., a dried substrate) to produce a second rehydrated reagent. The first rehydrated reagent and the second rehydrated reagent are then sequentially delivered to the detection area by capillary-driven flow to perform the assay.

### CROSS-REFERENCE TO RELATED APPLICATIONS

This application is related to and claims priority under 35 U.S.C. § 119(e) from U.S. Patent Application No. 63/058,683 filed Jul. 30, 2020 and titled “Automated Disposable Enzyme-Linked Immunosorbent Assay,” the entire contents of which is incorporated herein by reference for all purposes.

### GOVERNMENT LICENSE RIGHTS

This invention was made with government support under grant number HL152405 awarded by NIH and award number 2032222 by NSF. The government has certain rights in the invention.

### TECHNICAL FIELD

Aspects of the present disclosure generally relate to assay devices, and more particularly, to assay devices including microfluidic channels and colorimetric testing components.

### BACKGROUND

Capillary-driven microfluidic devices have gained popularity in the last decade as alternatives to traditional microfluidics. Instead of using an external pump to induce flow, capillary-driven devices utilize the surface tension of a fluid acting on channel walls (e.g., hydrophilic channel walls or fibers in the case of paper) to drive flow. Without the need for a pump, these devices can be operated outside of a centralized lab in resource limited settings without a power source, among other advantages. Pregnancy tests are just one example of capillary-driven analytical devices and their widespread utility as platforms for at-home diagnostics.

Immunoassays are a widely used technology for applications ranging from clinical diagnostics to environmental monitoring. The basis of the immunoassay is the binding reaction between antigen and antibody, typically performed on a surface. Either the antigen or the antibody can be the target analyte. After this reaction, the presence of the analyte is detected by one of several methods, including colorimetry, electrochemistry, fluorescence, and chemiluminescence. Immunoassays unfortunately rely heavily on laboratory instrumentation and thus, do not work well at the point-of-care or point-of-need. A related technology, the lateral flow assay (LFA) simplifies workflow but lacks the sensitivity and specificity of traditional immunoassays.

Considering the foregoing, a need exists for testing devices with the ease of use of LFAs and similarly immunoassays and the increased sensitivity associated with assays that conventionally require laboratory instrumentation.

### SUMMARY

In one aspect of the present disclosure, an assay device includes a colorimetric testing assembly including a detection area, a fluid inlet, and a microfluidic network. The microfluidic network includes a first path extending to the detection area and a second path extending to the detection area. The assay device further includes a first dried reagent disposed along the first path and a second dried reagent disposed along the second path. When a fluid is provided to the fluid inlet, a first portion of the fluid rehydrates the first dried reagent to produce a first rehydrated reagent, a second portion of the fluid rehydrates the second dried reagent to produce a second rehydrated reagent, and the first rehydrated reagent and the second rehydrated reagent are sequentially delivered to the detection area by capillary-driven flow.

In certain implementations, the assay device includes a pad. The pad may be disposed within the first path and contain the first dried reagent, or the pad may be disposed within the second path and contains the second dried reagent.

In another implementation, the first path includes a first surface on which the first dried reagent is disposed, or the second microfluidic path includes a second surface on which the second dried reagent is disposed.

In certain implementations, when fluid is provided to the fluid inlet, the first rehydrated reagent arrives at the detection area before the first rehydrated reagent.

In another implementation, the assay device includes a sample inlet separate from the fluid inlet and in communication with the microfluidic network. The sample inlet may include a filtration membrane.

In yet another implementation, the first path may be shorter than the second path.

In another implementation, the microfluidic network is configured such that the fluid arrives at the detection area by the first path before the fluid arrives at the detection area by the second path.

In still another implementation, the assay device includes a body formed from alternating layers of film and double-sided adhesive and the alternating layers of film and double-sided adhesive form the microfluidic network.

In another implementation, the assay device includes a vent in communication with the microfluidic network.

In another implementation, the assay device includes a second colorimetric testing assembly including a second detection area and a second microfluidic network in communication with each of the fluid inlet and the second detection area.

In another aspect of the present disclosure, a method of performing a colorimetric assay includes receiving a fluid at an inlet of an assay device. The assay device includes a microfluidic network in communication with each of the fluid inlet and a colorimetric assembly including a detection area. The method further includes rehydrating a first dried reagent disposed along a first path of the microfluidic network to produce a first rehydrated reagent, rehydrating a second dried reagent disposed along a second path of the microfluidic network to produce a second rehydrated reagent, and sequentially delivering the first rehydrated reagent and the second rehydrated reagent to the detection area by capillary flow.

In certain implementations, the assay device includes a device body formed from alternating layers of film and double-sided adhesive, and the alternating layers of film and double-sided adhesive define the microfluidic network.

In other implementation, the first rehydrated reagent includes an enzyme label, second rehydrated reagent includes a substrate, and the enzyme label is delivered to the detection area before the substrate.

In still other implementations, the fluid is a buffer fluid, and the method further includes receiving a sample at a sample inlet of the assay device, the sample inlet being in communication with the microfluidic network and separate from the fluid inlet. In such implementations, the method may further include transporting the sample by capillary action to the detection area such that the sample arrives at the detection area before each of the first rehydrated reagent and the second rehydrated reagent.

In certain implementations, the method may further include receiving a sample at a sample inlet of the assay device, the sample inlet in communication with the microfluidic network, and filtering the sample using a filtration membrane of the sample inlet. Such implementations may further include transporting the sample by capillary action to the detection area such that the sample arrives at the detection area before each of the first rehydrated reagent and the second rehydrated reagent.

In other implementations, the method may further include transporting a wash portion of the fluid by capillary action to the detection area before delivery of at least one of the first rehydrated reagent and the second rehydrated reagent to the detection area.

In another implementation, the method may include transporting a wash portion of the buffer fluid by capillary action to the detection area after delivery of the first rehydrated reagent and before delivery of the second rehydrated reagent.

In another aspect of the current disclosure, an assay device includes a colorimetric testing assembly including a detection area and a microfluidic network in communication with the colorimetric testing assembly. The microfluidic network is defined within a device body formed from alternating layers of film and double-sided adhesive. The assay device further includes a fluid inlet in communication with the microfluidic network, a first path extending to the detection area, and a dried enzyme label disposed along the first path. The assay device also includes a second path extending to the detection area, the second path being longer than the first path, and a dried substrate disposed along the second path.

In certain implementations, the fluid inlet is a buffer fluid inlet and the assay device further includes a sample inlet in communication with the microfluidic network.

### BRIEF DESCRIPTION OF THE DRAWINGS

The patent application file contains at least one drawing executed in color. Copies of this patent or patent application publication with color drawing(s) will be provided by the Office upon request and payment of necessary fee.

The present disclosure is described in conjunction with the appended figures.

FIG. 1 is a schematic illustration of an enzyme linked immunosorbent assay (ELISA).

FIG. 2 is a schematic illustration of an assay device according to the present disclosure.

FIGS. 3 and 4 are cross-sectional views of the assay device of FIG. 2.

FIGS. 5A-D are schematic illustrations of the assay device of FIG. 2 illustrating operation of the assay device.

FIGS. 6A-C are photographs illustrating operation of a prototype assay device substantially consistent with the assay device of FIG. 2.

FIG. 7 is a schematic illustration of a second assay device according to the present disclosure.

FIG. 8 is an exploded view of a device body of the assay device of FIG. 7.

FIGS. 9A-E are photographs of a prototype assay device consistent with the assay device of FIG. 7 during operation of the prototype assay device, with each including a respective illustration showing a state of a test strip.

FIG. 10 is a graph comparing performance of multi-inlet assay devices according to the present disclosure with conventional ELISAs for serology testing.

FIG. 11 are photographs comparing test strips for antigen tests performed using a single-inlet assay device according to the present disclosure and a conventional lateral flow assay.

FIG. 12 is a graph comparing performance of single-inlet assay devices according to the present disclosure with conventional ELISAs for antigen testing.

FIG. 13 is a photograph illustrating an example prototype device including a single-inlet assay device according to the present disclosure.

FIG. 14 is a schematic illustration of a multi-assay device.

FIG. 15 is a flow chart illustrating a method of performing an assay requiring separate introduction of a sample and buffer according to the present disclosure.

FIG. 16 is a flow chart illustrating a method of performing an assay of a combined sample/buffer fluid.

In the appended figures, similar components and/or features can have the same reference label. Further, various components of the same type can be distinguished by following the reference label by a dash and a second label that distinguishes among the similar components. If only the first reference label is used in the specification, the description is applicable to any one of the similar components having the same first reference label irrespective of the second reference label.

### DETAILED DESCRIPTION

Aspects of the present disclosure are directed to capillary-driven microfluidic devices and, more specifically, such devices for performing colorimetric testing. In general, the devices disclosed herein include a microfluidic network through which fluid is transported by capillary action and that automatically sequences the delivery of reagents and washes to a test/detection zone. Sequencing is achieved, at least in part, by varying the geometry and/or other flow driving characteristics of pathways through the microfluidic network. For example, the microfluidic network may include paths of varying length between a buffer inlet and the detection zone such that fluid transported along longer paths is delivered to the detection zone after fluid transported along shorter paths. Dried reagents may be disposed along certain paths of the microfluidic network such that the reagents may be rehydrated and delivered to the detection zone. The flow-driving characteristics of the paths (e.g., the geometry of the paths) relative to each other may therefore be used to control the timing and sequencing of delivery of the rehydrated reagents. Flow through the devices and subsequent sequencing of reagent delivery to the detection zone is substantially automatic and generally requires only that a user provide each of a sample and buffer solution or a combined sample and buffer to the device, depending on the particular test to be conducted using the device.

Enzyme linked immunosorbent assays (ELISAs) are used to detect a wide range of analytes with good sensitivity and specificity. These analytes include whole cells, proteins, antibodies, and small molecules, among other things. The high sensitivity of ELISAs is enabled by stringent washing steps to mitigate non-specific adsorption of non-targets and using an catalytic label such as an enzyme to amplify a signal, while the high specificity is enabled using a “sandwich” immunoassay that captures an analyte between two highly specific probes.

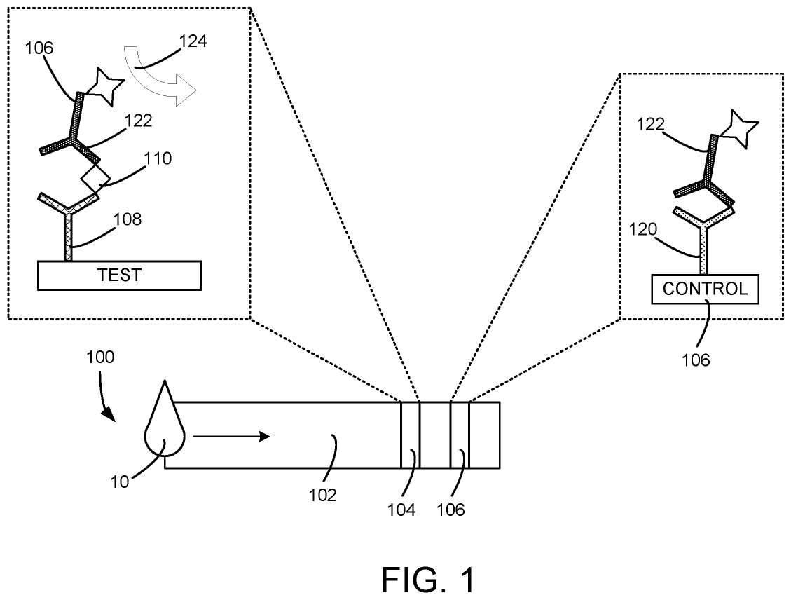

An example of a conventional ELISA system 100 is illustrated in FIG. 1. The ELISA system 100 includes a strip 102 that may have one or more detection zones or lines. In the specific example of system 100, the lines include a test line 104 and a control line 106. The strip 102 may be formed from nitrocellulose or a similar material such that, when a sample 10, is disposed on strip 102, sample 10 is wicked or otherwise transported to test line 104 and control line 106.

Control line 106 may be optional but is generally included in ELISA systems to verify proper device functionality. In general, control line 106 includes an anti-label 120 adapted to bond with an enzyme label 122. Test line 104, on the other hand, includes a capture probe 108 configured to bond with a target analyte 110, to which enzyme label 122 bonds. Following delivery of target analyte 110 and enzyme label 122, an enzyme substrate 124 is provided that reacts with enzyme label 122 to produce a product and a corresponding colorimetric or other visual change of the corresponding line.

Both enzymatic detection and the sandwich assay format require multiple washing steps and sequential delivery of reagents (e.g., the enzyme label and the substrate). These washing and reagent delivery steps yield robust analytical performance from ELISAs, but they are complicated to perform and relegate the assay to a centralized laboratory with expensive equipment and trained laboratory technicians. In many testing situations the time and financial resources needed for ELISAs are not available. There are more affordable point-of-care alternatives, but they often lack the analytical performance needed.

One of the most common alternatives to an ELISA is the lateral flow immunoassay. Lateral flow strips are inexpensive and easy to use in comparison to the ELISA. They generally require only that the end user provide a sample and the results are easy to interpret in most settings. The pregnancy test is the most widespread example of a lateral flow device, having an estimated global market of $1.3 billion per year. Although they are easy to use, lateral flow strips perform poorly compared to ELISAs, with well-documented reports of high false positive and negative rates and unreliable quantification. Substandard sensitivity and specificity in lateral flow assays stem from the inability to use enzymes as labels and sequentially wash and add reagents, which limits the scope of possible target analytes (e.g. disease biomarkers, antibodies, whole cells).

To address the foregoing issues, among others, this disclosure introduces a system that can be as easy to use as a pregnancy test or other lateral flow assay, but with the same analytical capabilities of an ELISA. The system relies on a capillary driven immunoassay device to sequentially add and wash reagents and competing/non-target species from a test zone with minimal steps by an end user. The assay device generally includes a microfluidic network with channels that are specifically configured to provide sequential flow of reagents and washing fluids to a detection zone. Operation of the assay device is substantially automatic, with an end user only being required to provide a sample and a buffer fluid or a sample/buffer combination. In certain implementations, flow through the assay device is achieved by capillary driven flow, which may be facilitated by hydrophilic channels of the microfluidic network and a passive pump mechanism, such as a paper/nitrocellulose pump. Notably, assay devices according to the present disclosure are capable of performing tests similar to conventional ELISAs, enabling quick and accurate detection of target analytes in at-home and other settings and by untrained users that were previously undetectable outside a centralized laboratory.

In certain implementations, the assay device is made of film sheets (e.g., transparency sheets, polyester film) and double-sided adhesive layers. Each layer may be laser cut or otherwise manufactured such that, when the layers are assembled by stacking and laminated, a microfluidic network is defined within the resulting laminated body.

The microfluidic network includes multiple channels/paths, each configured to transport fluid (e.g., a buffer fluid) by capillary flow from one or more fluid inlets to a testing area/detection zone. Certain of the channels may include dried reagents that are rehydrated by fluid transported through the channels for subsequent delivery to the testing area. Other channels may not include reagents such that fluid transported through such channels is delivered to the testing area without substantially altering the composition of the fluid. As a result, fluid transported through channels furnished with a dried or otherwise stabilized reagent may be used to deliver the reagent to the testing area. Washes may be provided, for example, by fluid portions that do not include rehydrated reagent or that are provided by other paths/channels of the microfluidic network that do not include dried reagents. By varying the length, size, and similar characteristics of the channels of the microfluidic network and including other flow control mechanisms, delivery of fluid via different channels to the testing area/detection cone may be sequenced. So, for example, delivery of reagents and washes may be alternated or otherwise sequenced to follow a particular testing protocol.

Dried reagents may be provided by conjugate release pads disposed along channels of the microfluidic network. Such pads may be formed, e.g., from glass fiber or nitrocellulose. Alternatively, dried reagent may be disposed along a given channel, e.g., by being dried onto a surface of the channel.

In certain implementations, the testing area may be provided by a nitrocellulose membrane connected to or otherwise in communication with the microfluidic network. The nitrocellulose membrane may contain a capture probe specific to the target analyte. The capture probe may be striped onto the nitrocellulose membrane to form a test line. During operation, an enzyme-labeled detection antibody specific to the target analyte may be delivered to the test line (e.g., by rehydrating a dried antibody disposed in a channel of the microfluidic network) followed by subsequent delivery of a substrate (e.g., by rehydrating a dried antibody disposed in a channel of the microfluidic network) that reacts with the enzymatic label to produce a colorimetric signal at the test line.

The assay device may include a specific sample inlet and related components for processing a sample for testing, such as a filtration membrane for filtering the sample. When performing an assay based on whole blood, for example, the filtration membrane may be a plasma separation membrane and may be sealed over the sample inlet. In such cases, the assay device may further include a separate fluid/buffer inlet for introducing a fluid buffer to initiate subsequent testing of the sample. For less viscous or less complex sample matrices (e.g., nasal swab samples diluted in an extraction buffer) a single sample inlet/buffer inlet with no filtration membrane could be used. In such application, a separate membrane or similar element may not be necessary, and the sample may also supply the buffer for the rest of the assay. In general, however, the same reagent addition and washing steps may be accomplished with single-inlet assay device as with multi-inlet devices by configuring the microfluidic network accordingly.

Example implementations discussed herein focus on non-competitive immunoassays; however, implementations of the present disclosure are not limited to such assays. Rather, this disclosure is intended to describe a more general assay device that may be used in a range of applications. Stated differently, this disclosure is intended to describe a general assay device capable of sequential delivery of reagents and washes to a colorimetric test assembly. Although specific tests (including specific sample types, reagents, buffers, etc.) may be described, such tests should be considered illustrative only and non-limiting regarding other applications for the present disclosure. For example, while most examples discussed herein focus on testing of bodily fluids, assay devices according to this disclosure may be adapted for environmental or chemical testing (e.g., water testing). As another example, while the examples disclosed herein generally focus on non-competitive immunoassays, assay devices according to the present disclosure may be adapted to perform competitive immunoassays. Competitive immunoassays are more commonly used for small molecules, like hormones, THC, and other small molecular weight molecules where an antibody pair is not available. In a competitive immunoassay, a capture antibody is deposited in a detection area (e.g., on a nitrocellulose strip). A detection reagent in the system would be a labeled version of the analyte (e.g., the analyte labelled with an enzyme, metal, nanoparticle, etc.). When the sample runs through the system with no analyte, the labeled detector analyte binds to the capture antibody and gives a signal, e.g., a color change at the test line. If analyte is present in the sample, it competes with the detector analyte binding at the detection area and reduces the resulting signal. Therefore, a reduction in signal is observed in the presence of specific analyte. So, for example, in implementations of the present disclosure, an assay device may be configured to deliver a capture antibody to a detection area followed by a sample, followed by a labeled version of the analyte, with optional washes between each delivery. More generally, while specific assay devices are described herein, such devices may be readily adapted for different applications by modifying the placement and type of reagents included in the assay device as required for the assay to be performed.

The foregoing introduces certain concepts related to assay device devices. Other features and aspects of such devices and related technology are described below with reference to various example implementations.

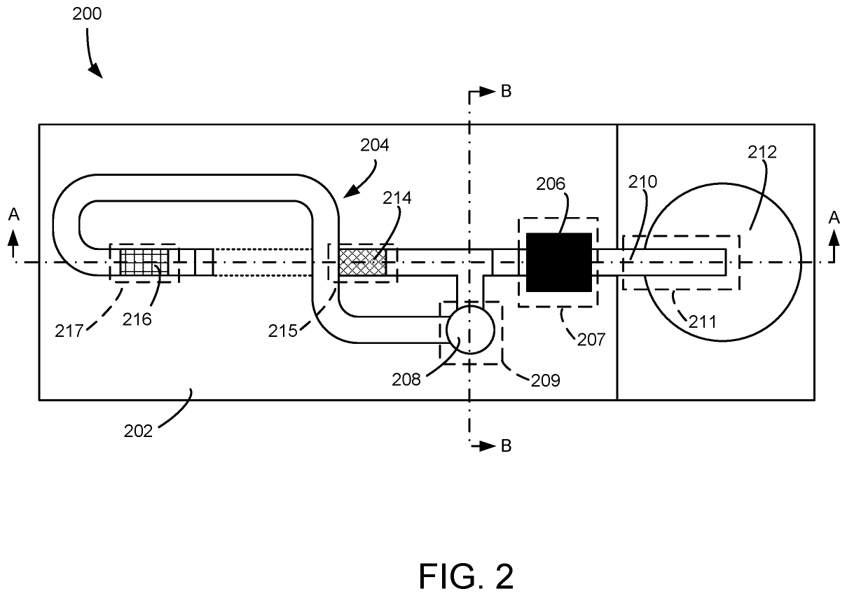

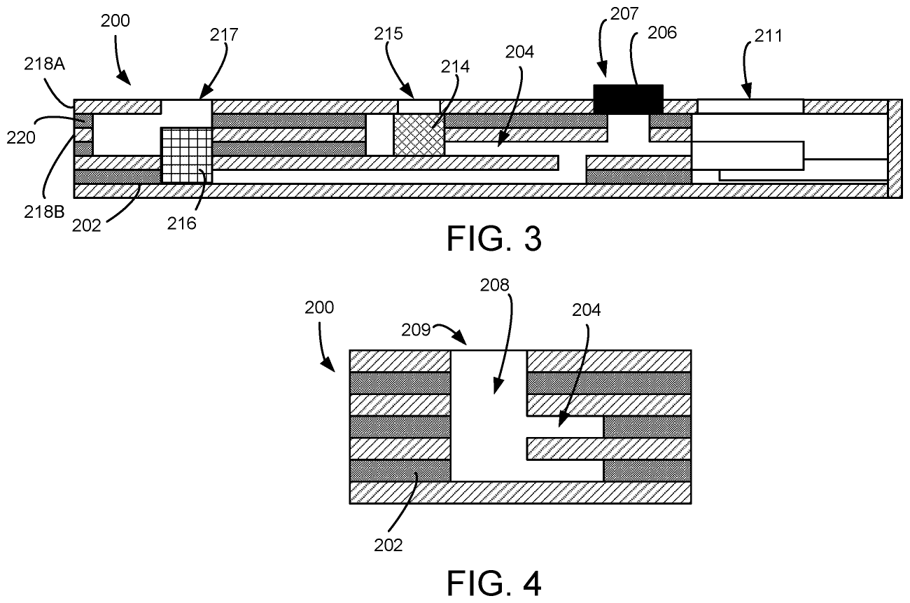

FIGS. 2-4 illustrate a first implementation of an assay device 200 according to the present disclosure. More specifically, FIG. 2 is a plan view of assay device 200 while FIGS. 3 and 4 are cross-sectional views taken along A-A and B-B, respectively.

As illustrated, assay device 200 includes a device body 202 defining a microfluidic network 204. Assay device 200 includes a sample inlet 206 and a buffer inlet 208 in communication with microfluidic network 204. Microfluidic network 204 generally includes microfluidic pathways or channels for transporting fluids provided via sample inlet 206 and buffer inlet 208 to a test strip 210 (e.g., a colorimetric test strip). In general, the channels of microfluidic network 204 are configured to transport fluids by capillary action. Such transportation may be facilitated by forming device body 202 from or otherwise applying hydrophilic materials to surfaces of the channels of microfluidic network 204. Transportation may be further facilitated by a nitrocellulose or similar “wicking” substrate of test strip 210 alone or in combination with a passive pump 212. As shown in FIG. 2, passive pump 212 may be in the form of a waste pad that also collects excess fluid.

Assay device 200 is generally configured to perform a test like a conventional enzyme linked immunosorbent assay (ELISA). To facilitate such testing, assay device 200 includes dried reagents disposed along channels of microfluidic network 204. Specifically, assay device 200 includes a dried enzyme label pad 214 and a dried substrate pad 216 disposed within microfluidic network 204. In other implementations, assay device 200 may be adapted to perform other assays by changing, adding, removing, or otherwise modifying the specific reagents included in microfluidic network 204. Such modification may include adding additional paths of microfluidic network 204 for delivery of additional reagents.

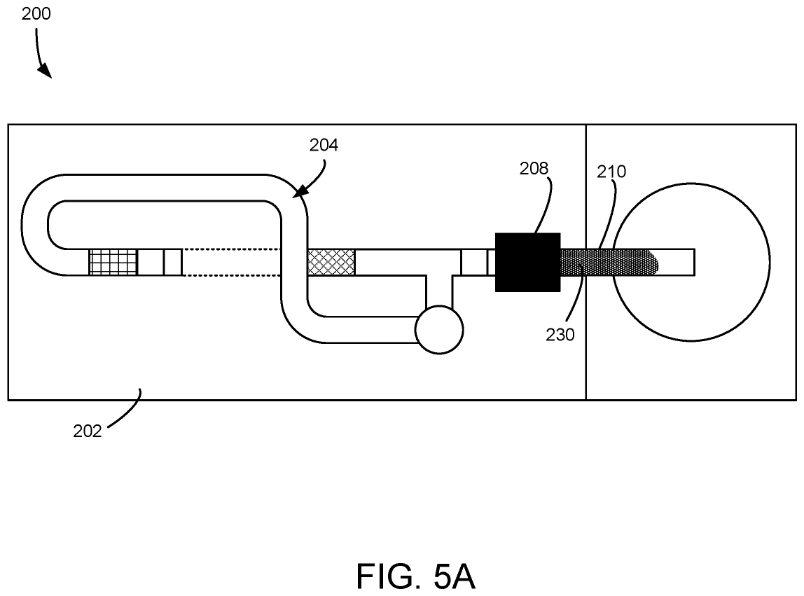

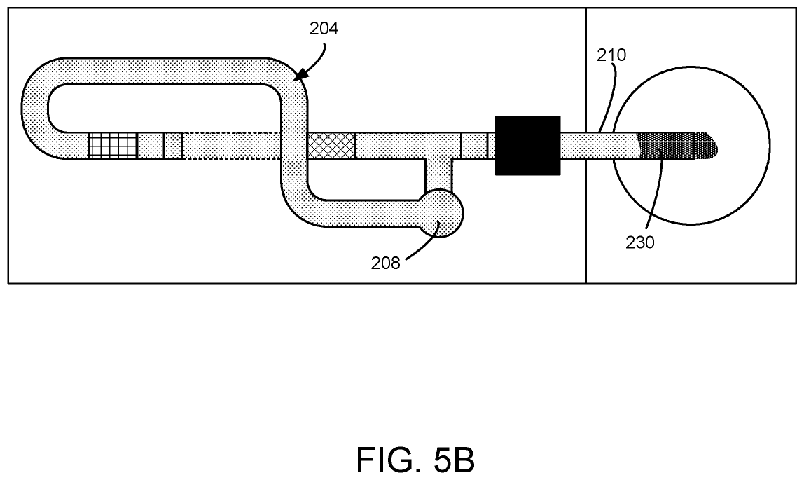

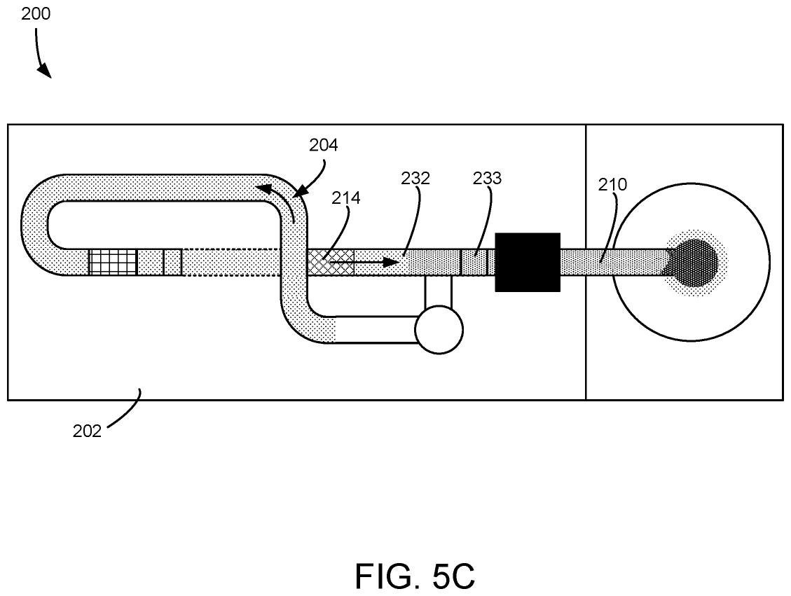

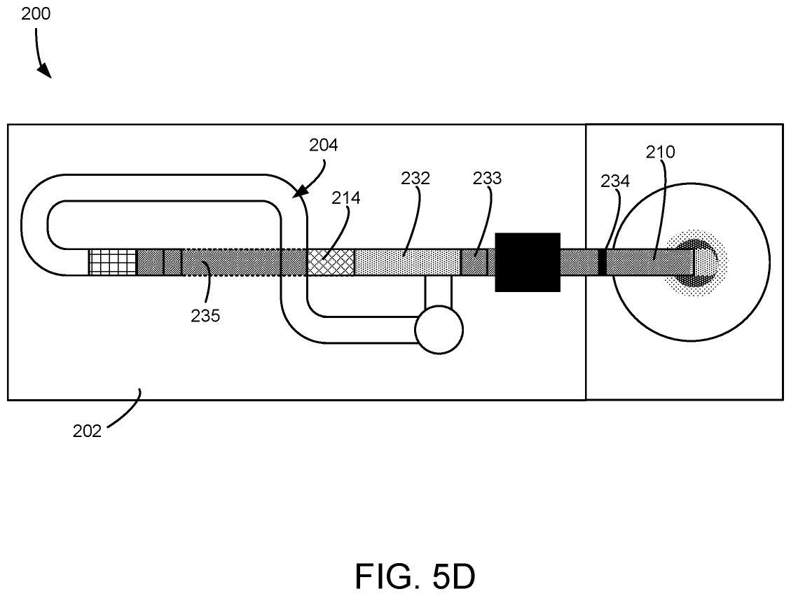

As described below in further detail in the context of FIGS. 5A-D, the configuration of assay device 200 is such that a sample, enzyme label, and substrate are each delivered to test strip 210 sequentially with intervening washes. More specifically, a user of assay device 200 provides a sample via sample inlet 206, which may be transported to test strip 210. The user subsequently provides a buffer fluid via buffer inlet 208, which flows through microfluidic network 204. Addition of the buffer fluid may further drive or transport additional sample to test strip 210 and, after delivery of the sample, may provide a wash of excess sample from test strip 210. The buffer fluid within the microfluidic network rehydrates the reagents on dried enzyme label pad 214 and dried substrate pad 216 and enables transport of the reagents to test strip 210. As illustrated in FIG. 2, dried enzyme label pad 214 is positioned within microfluidic network 204 relative to dried substrate pad 216 such that rehydrated enzyme label arrives at test strip 210 first. Due to the spacing between dried enzyme label pad 214 and dried substrate pad 216 a plug of buffer fluid is subsequently delivered, washing test strip 210 of excess enzyme label. As capillary driven flow continues, rehydrated substrate from dried substrate pad 216 is delivered to test strip 210, generally completing the test process. After delivery of rehydrated substrate, a certain time may be required to elapse before a colorimetric change is observed at test strip 210, indicating the result of the assay. Implementations of the present disclosure are not limited to any specific enzyme label and the specific enzyme label used may vary based on the specific assay to be performed using assay device 200. For example, in at least certain implementations, the enzyme label may be either of a biological enzyme or a non-biological enzyme (e.g., a nanozyme).

In certain implementations, a sample may require processing as part of the testing process. In such cases, sample inlet may include a filtration membrane or similar component for processing the sample. For example, when testing blood, sample inlet 206 may include a plasma or similar membrane to separate blood components.

As previously noted, FIGS. 3 and 4 are cross-sectional views of assay device 200 along A-A and B-B, respectively. As shown, device body 202 may be formed from multiple laminated layers of material coupled together. For example, in certain implementations, device body 202 may be formed by alternating layers of film (e.g., film layers 218A, 218B) and double-sided adhesive material (e.g., double-sided adhesive layer 220), each of which may be cut (e.g., laser cut) or otherwise formed to produce microfluidic network 204. Accordingly, assembly of assay device 200 may include forming, stacking, and adhering layers of device body 202. During such assembly, additional components (e.g., dried enzyme label pad 214 and dried substrate pad 216) may be disposed within device body 202, as required. In other implementations, other manufacturing techniques may be used. For example, and without limitation, in at least certain implementations, device body 202 may be formed by 3D printing or related techniques.

In FIG. 2, assay device 200 is generally illustrated with certain portions of device body 202 selectively removed for better illustrating aspects of assay device 200 and, more specifically, microfluidic network 204. Accordingly, in at least certain implementations, device body 202 may include a topmost layer (e.g., layer 218A) that substantially covers and contains microfluidic network 204 and components disposed therein, as generally illustrated in FIGS. 3 and 4. Notably, the topmost layer may include various openings for providing various functions. In addition to openings to permit introduction of samples and/or buffer fluid (e.g., a sample inlet opening 207 corresponding to sample inlet 206 and a buffer inlet opening 209 corresponding to buffer inlet 208), the topmost layer may include vents or similar openings corresponding to each of dried enzyme label pad 214 and dried substrate pad 216. For example, device body 202 includes each of a first vent 215 for dried enzyme label pad 214 and a second vent 217 for dried substrate pad 216. General locations for each of the foregoing vents are also illustrated in long dashed lines in FIG. 2. As discussed below, such venting functions permit proper filling of assay device 200 with buffer fluid and generally preclude the formation of air bubbles that may negatively impact or disrupt flow through microfluidic network 204. The top layer may further include a test strip opening 211 for viewing results on test strip 210.

A discussion of the use of assay device 200 is now provided with reference to FIGS. 5A-D.

Referring first to FIG. 5A, a sample 230 is added to sample inlet 206. As previously noted, adding sample 230 to sample inlet 206 may include processing (e.g., filtering using a filtration membrane) by a corresponding component integrated into sample inlet 206. Sample inlet 206 is in communication with microfluidic network 204. As illustrated, at least a portion of sample 230 (which may be a filtered component of a whole sample) may be transported to test strip 210 by capillary action and further facilitated by a cellulose or similar passive pumping mechanism. In certain implementations, such pumping functionality may be provided by test strip 210, optional passive pump 212, or a combination therefore.

Referring next to FIG. 5B, a user adds a buffer fluid 232 via buffer inlet 208. Buffer inlet 208 is in communication with microfluidic network 204 such that adding buffer fluid 232 via buffer inlet 208 results in buffer fluid 232 being distributed substantially throughout microfluidic network 204 by capillary action. When added, at least a portion of buffer fluid 232 may further drive transport of sample 230 across test strip 210. After sample 230 is substantially driven across test strip 210, a plug/portion of buffer fluid 232 may follow, thereby washing excess of sample 230 from test strip 210.

FIG. 5C illustrates assay device 200 during delivery of rehydrated enzyme label from dried enzyme label pad 214. More specifically, after introduction of buffer fluid 232, reagent stored in dried enzyme label pad 214, at least a portion of a dried enzyme label stored on dried enzyme label pad 214 may be rehydrated by buffer fluid 232 and a resulting rehydrated enzyme label 233 may be subsequently transported through microfluidic network 204. Like the washing step performed when introducing buffer fluid 232, which resulted from a portion of buffer fluid 232 following sample 230 across test strip 210, another portion of buffer fluid 232 may follow rehydrated enzyme label 233 across test strip 210 to perform an additional washing step that removes excess of rehydrated enzyme label 233 from test strip 210.

As shown in FIG. 5D, buffer fluid 232 rehydrates dried substrate pad 216 to produce rehydrated substrate 235. Rehydrated substrate 235 is transported through microfluidic network 204 to test strip 210. In assay device 200, such transportation includes transporting rehydrated substrate 235 by capillary action along a path/channel of microfluidic network 204 that extends through a lower layer of device body 202 and that emerges upstream of test strip 210. When rehydrated substrate 235 ultimately arrives at test strip 210, it reacts with the previously delivered rehydrated enzyme label 233 and sample 230, resulting in a colorimetric change in the test strip 210 (e.g., the appearance of a stripe 234).

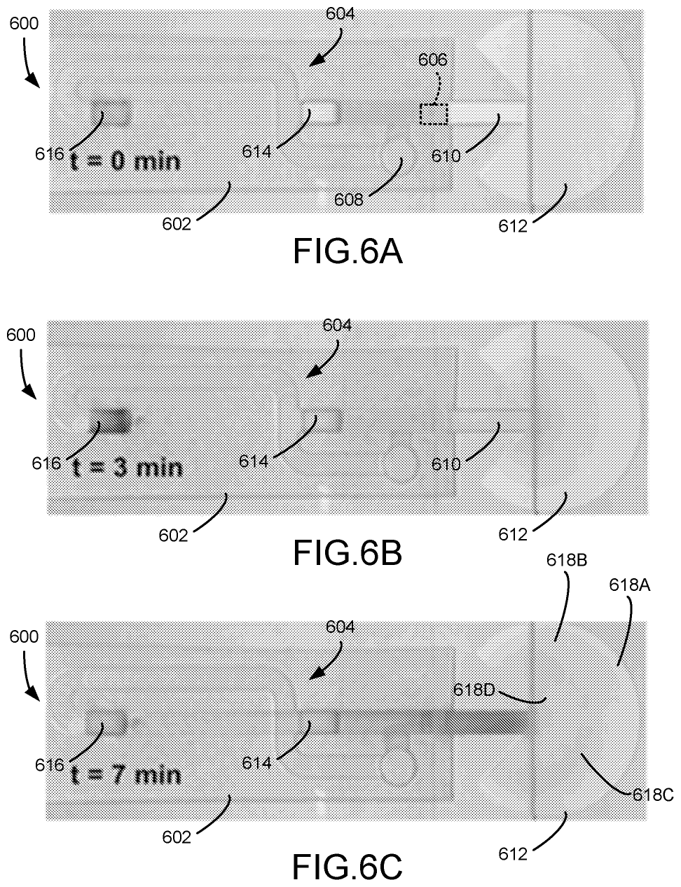

FIGS. 6A-C are photographs of a prototype assay device 600 further illustrating the previous description. Like assay device 200, assay device includes a device body 602 defining a microfluidic network 604. Assay device 600 also includes a buffer inlet 608 in communication with microfluidic network 604. Microfluidic network 604 transports fluids provided via buffer inlet 608 to a test strip 610, which is shown as being coupled to a waste pad/cellulose pump 612. Although not included in the depicted prototype, assay device 600 may also include a sample inlet 606 (general location shown in dotted lines) for introduction and potential processing (e.g., separation) of a sample to be tested using assay device 600. In FIGS. 6A-C food coloring was generally used in place of an actual enzyme label and substrate to make flow through assay device 600 clearer and more comprehendible; however, the foregoing discussion nevertheless refers to an enzyme label and substrate.

FIG. 6A illustrates the assay device 600 at an initial time (t=0), i.e., just before or immediately on introduction of a buffer fluid via buffer inlet 608. FIG. 6B illustrates the assay device 600 between delivery of a rehydrated enzyme label and a rehydrated substrate. As previously discussed in the context of FIGS. 5A-D, introduction of a buffer fluid to buffer inlet 608 causes the buffer fluid to be transported throughout microfluidic network 604. When distributed through microfluidic network 604, the buffer fluid begins rehydrating an enzyme label stored on dried enzyme label pad 614 and a substrate stored on dried substrate pad 616. The rehydrated reagents are then transported to test strip 610 by capillary action. Given that dried enzyme label pad 614 is positioned substantially closer to test strip 610 than dried enzyme label pad 614 and as illustrated in FIG. 6B, rehydrated enzyme from dried enzyme label pad 614 is delivered to test strip 610 substantially earlier than rehydrated substrate from dried substrate pad 616. After delivery of the rehydrated enzyme label, the rehydrated substrate may be delivered to test strip 610 as shown in FIG. 6C. Although FIG. 6C was capture prior to a reaction at test strip 610, delivery of the rehydrated substrate to test strip 610 would generally result in a colorimetric change to test strip 610 (e.g., the appearance of a line) indicating the presence of the target analyte, if present.

Notably, and as previously discussed in the context of FIGS. 5A-C, each of sample delivery and delivery of the rehydrated enzyme label may be followed by a portion/plug of the buffer fluid to wash excess sample and enzyme label from test strip 610, respectively. Evidence of such sequencing can be seen on cellulose pump/waste pad 612 of FIG. 6C and the pattern of fluid thereon. Specifically, cellulose pump/waste pad 612 generally includes multiple bands corresponding to fluids absorbed by cellulose pump/waste pad 612 and the sequence that such fluids were absorbed. A first, outermost band 618A corresponds to an unsaturated portion of cellulose pump/waste pad 612. A second band 618B is darker, indicating absorption of the buffer fluid. A third band 618C is the color of the enzyme label, indicating subsequent delivery of the enzyme label. Finally, a fourth band 618D is light, indicating another delivery of the buffer fluid (e.g., for washing excess enzyme label).

The sequential reagent delivery and washing illustrated in FIGS. 5A-6C is facilitated by varying the geometries of flow paths through the microfluidic networks. For example, all other geometries being equal, fluid transported along a first path of a microfluidic network will arrive before fluid transported along a second path of the microfluidic network when the second path is longer. Accordingly, the first path may be used to deliver a first reagent to a test strip at a first time and the second path may be used to deliver a second reagent to the test strip thereafter. Similar results may be achieved by altering other geometric characteristics (e.g., surface area, cross-section shape, etc.) of flow paths within the microfluidic network, forming flow paths from different materials, coating surfaces of the flow paths with different materials, and the like. For example, a first path may include a surface that is more hydrophilic then a surface of a second path to drive capillary flow. In such cases, fluid transported along the first path will generally arrive at a destination prior to fluid transported along the second path, all other things being equal.

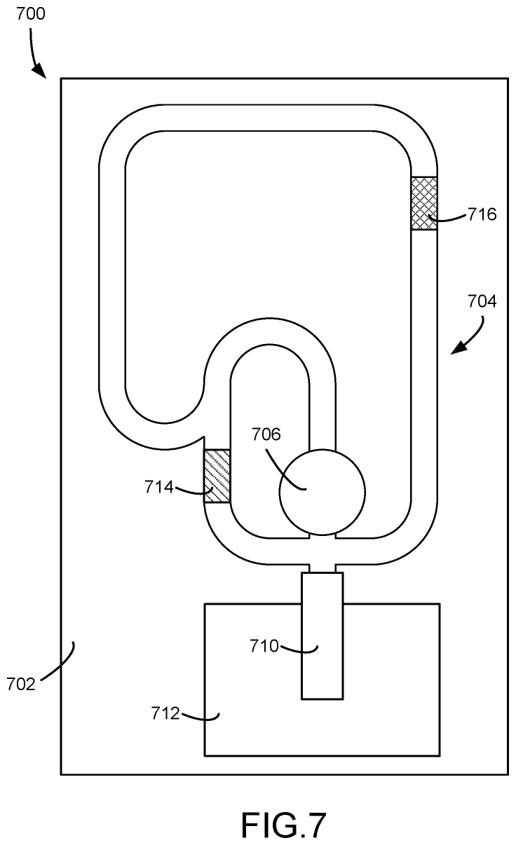

FIG. 7 is a schematic illustration of another assay device 700 according to the present disclosure. Assay device 700 includes a device body 702 defining a microfluidic network 704. In contrast to assay device 200, which included each of a sample inlet 206 and a buffer inlet 208, assay device 700 includes only a single inlet 706 in communication with microfluidic network 704. As previously noted, dual- or multi-inlet assay device devices may be useful when a sample requires separate processing, such as filtering through a filtration membrane. Single-inlet designs, on the other hand, may be suitable for applications having less viscous or complex sample matrices that may be mixed with a buffer solution and provided simultaneously to the assay device. For example, single-inlet devices may be suitable for use in testing nasal swab samples diluted in an extraction buffer. In general, microfluidic network 704 transports fluids provided via inlet 706 to a test strip 710, which is shown as being coupled to a waste pad/pump 712. Assay device 700 is generally configured to perform a test including enzyme labelling and subsequent application of a substrate. Accordingly, assay device 700 includes a dried enzyme pad 714 and a dried substrate pad 716 disposed within microfluidic network 704.

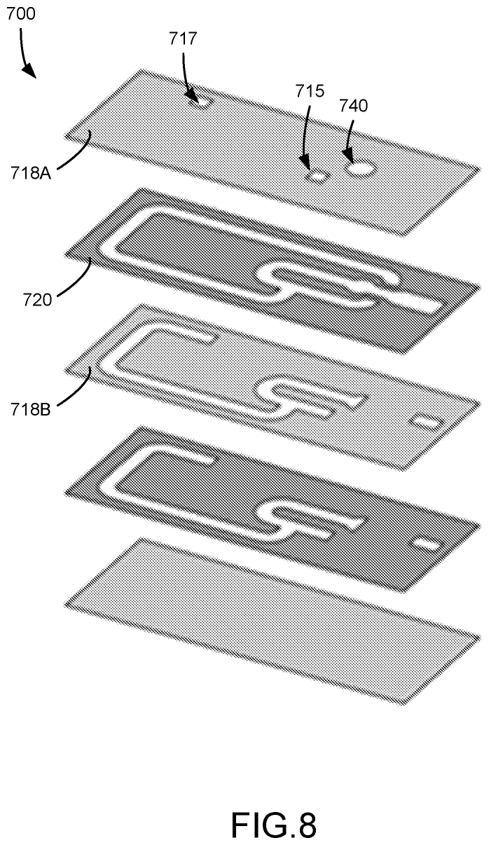

FIG. 8 is an exploded view of assay device 700. Like assay device 200, assay device 700 may be formed from multiple layers including from alternating layers of film (e.g., film layers 718A, 718B) and double-sided adhesive (e.g., double-sided adhesive layer 720). Alternatively, assay device 700 may be 3D printed or manufactured using any other suitable techniques.

As illustrated in FIG. 8, film layer 718A, which corresponds to a topmost layer of assay device 700, may define various openings. More specifically, film layer 718A includes each of an inlet opening 740 corresponding to inlet 706 (shown in FIG. 7) and each of a first vent 715 corresponding to dried enzyme label pad 714 (shown in FIG. 7) and a second vent 717 corresponding to dried substrate pad 716 (shown in FIG. 7). As noted above in the context of assay device 200, vents 715, 717 generally facilitate filling of device body 702 with buffer fluid without formation of bubbles that may disrupt flow through microfluidic network 704.

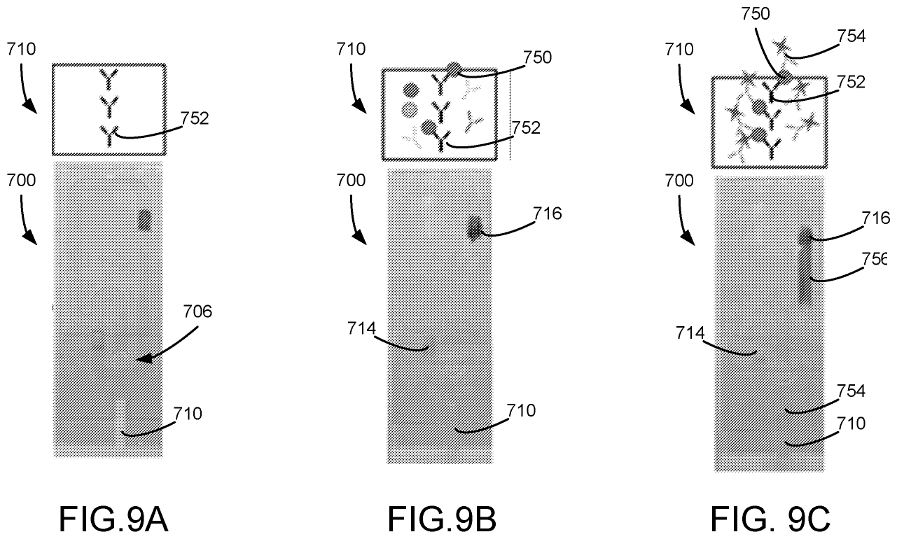

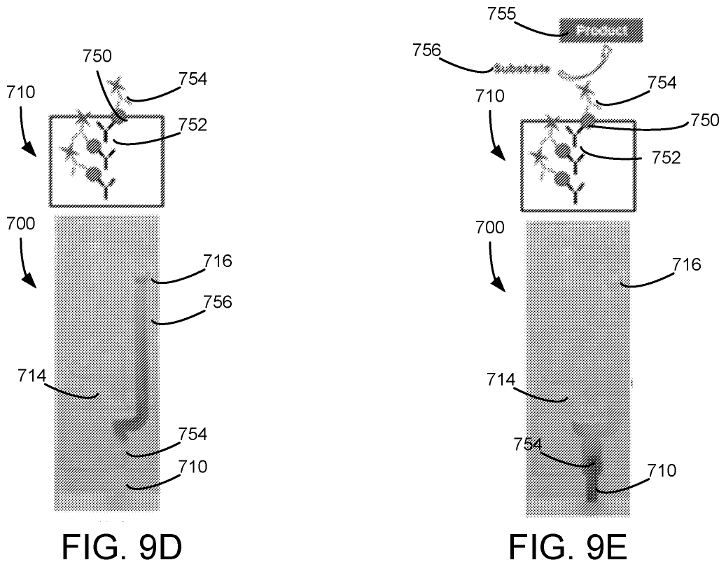

FIGS. 9A-E illustrate operation of assay device 700. More specifically, each of FIGS. 9A-E include a photograph of assay device 700 accompanied by a schematic illustration of test strip 710 that indicating the stat of test strip 710 and any reagents/substances provided thereto. Like the example illustrated in FIGS. 5A-D, food coloring was used in place of actual enzyme labels and reagents to better illustrate operation of assay device 700; nevertheless, the following example still refer to enzyme labels and substrates.

FIG. 9A illustrates assay device 700 in a pre-assay state. In the pre-assay state, fluid has not yet been provided to inlet 706 and each of enzyme label pad 714 and dried substrate pad 716 include corresponding dried reagents. As illustrated in the detailed view of test strip 710, test strip 710 may include capture probes (such as capture probe 752) adapted to bond with a target analyte. As previously discussed, test strip 710 may include other capture probes for purposes of testing and validating operation of assay device 700.

FIG. 9B illustrates assay device 700 following addition of a test fluid to inlet 706. In general, the fluid is distributed from inlet 706 to different channels of microfluidic network 704 by capillary action. Such distribution of the test fluid may result in the test fluid contacting dried enzyme label pad 714 and initiating rehydration of the dried enzyme label stored on dried enzyme label pad 714. Similarly, a portion of the test fluid may also contact dried substrate pad 716, initiating rehydration of a dried substrate. Yet another proportion of the fluid is transported to test strip 710 such that a proportion of a target analyte 750 contained within the test fluid bonds with the capture probes 752. As illustrated in the detailed view of test strip 710 included in FIG. 9B, introduction of the test fluid may cause other analytes and trace amounts of the rehydrated enzyme label and rehydrated substrate to reach test strip 710.

FIG. 9C illustrates assay device 700 following introduction of the test fluid and substantial elapsed time such that a substantial portion of the rehydrated enzyme label 754 is transported from dried enzyme label pad 714 to test strip 710. As further illustrated in FIG. 9C, a portion of rehydrated substrate 756 has simultaneously started moving toward test strip 710. Also, as illustrated in the detail view of test strip 710 included in FIG. 9C, at least a portion of rehydrated enzyme label 754 arriving at test strip 710 bonds with target analyte 750 previously captured at test strip 710, thereby preparing test strip 710 for delivery of rehydrated substrate 756.

During delivery of enzyme label from dried enzyme label pad 714, an excess of enzyme label may be delivered to test strip 710. Accordingly, FIG. 9D illustrates assay device 700 during a wash after delivery of rehydrated enzyme label 754. More specifically, microfluidic network 704 is generally shaped and configured such that at least a portion of the test fluid is delivered to test strip 710 after rehydrated enzyme label 754 but before rehydrated substrate 756. By doing so, excess of the enzyme label can be removed from test strip 710 before arrival of rehydrated substrate 756, generally improving the response of test strip 710.

FIG. 9E illustrates assay device 700 during delivery of rehydrated substrate 756 to test strip 710. As shown in the inset of FIG. 9E, delivery of rehydrated substrate 756 results in the production of a product 755, which may, in turn, cause a colorimetric change in test strip 710, such as the appearance of a colored strip.

Notably, operation of any of the foregoing devices and others in accordance with the present disclosure is straightforward from the perspective of an end user and substantially automated. More specifically, in double- or multi-inlet assay device devices, a sample is added to an inlet (which may include a filtration membrane or similar element) and buffer is subsequently added to a buffer inlet. The addition of the buffer starts the sequential reagent delivery and washing cycles without any further intervention by the user. Accordingly, the only substantive steps to be performed by the end-user to execute an assay are the addition of the sample and buffer. Similarly, in single-inlet devices, the assay is initiated by adding a combined sample and buffer (e.g., a sample diluted in a sample buffer) to an inlet. Accordingly, the only step to be performed by the end-user to perform the assay is the addition of the sample and buffer to the assay device.

As previously discussed, in at least certain implementations, after sample and buffer addition, all channels of the microfluidic network may be filled with buffer (or combined buffer and sample) due to capillary action. Notably, the microfluidic network may include vents or similar openings (e.g., above the dried reagent pads) to ensure proper filling of the assay device and to ensure venting of air to prevent bubbles that may impede flow through the microfluidic network. Once the channels are filled, passive pump (e.g., a waste pad, nitrocellulose body of a test strip, etc.) may be coupled to the microfluidic network or otherwise made to contact the fluid within the microfluidic network, thereby pumping/drawing fluid through the microfluidic network to a testing or detection zone (e.g., of a test strip). In at least certain implementations, the sample inlet is placed immediately upstream of the detection zone such that the sample is delivered to the detection zone first.

Assay devices according to this disclosure may include two or more dried reagents, which may be stored on pads within the microfluidic network or otherwise disposed within the microfluidic network. In certain implementations, a first of the dried reagents may be an enzyme or nanozyme label while a second of the dried reagents may be a substrate. The microfluidic network is generally configured such that after introduction of a sample, the sample may be transported to the detection zone by capillary action. The user may then introduce a buffer fluid to the assay device, which substantially fills the microfluidic network and, in some instances, further drives flow of the sample to the detection zone. A portion of the buffer fluid may follow, thereby washing away excess sample that may interfere with the remaining assay. Following introduction of the buffer fluid, dried reagent stored within the microfluidic network may be rehydrated by the buffer fluid and generally permitted to flow toward the detection zone. In a two-reagent configuration, the pressure differential or other parameter impacting flow is such that rehydrated reagent from the first reagent pad (e.g., enzyme label) arrives at the detection zone before rehydrated reagent from the second reagent pad. As a result, target analyte captured in the detection zone may captures the rehydrated reagent form the first reagent pad.

Following delivery of the first reagent, the second reagent (e.g., rehydrated substrate) may be delivered to the detection zone. The rehydrated substrate may be preceded at the detection zone by additional buffer fluid, which washes away excess of the first reagent. When the rehydrated second reagent reaches the detection zone, the rehydrated second may react with the first reagent, producing a visible color change or similar effect.

In certain implementations, after flow is substantially complete in the device, the color change may generally be detectable with the naked eye for qualitative detection, imaged (e.g., using a smartphone camera) for quantitative information, or otherwise interpreted.

As noted above, FIGS. 6A-C and FIGS. 9A-D are photographs illustrating example operation of dual-inlet assay device 600 and single-inlet assay device 700 using food dye/food coloring. Notably, after verifying operation of assay device 600, a prototype dual-inlet assay device like assay device 600 was tested for an immunoassay for anti-SARs-CoV-2 antibodies. Similarly, a single-inlet assay device based on the design of assay device 700 was used to conduct a SARs-CoV-2 nucleocapsid protein (N protein) assay. Serology tests look for the presence of antibodies in blood to determine disease progression and/or confirm an individual had been infected with a particular pathogen. Because blood is the standard sample type for serology assays, a two-inlet assay device with a plasma separation membrane, such as assay device 600, was required for the serology test (i.e., the test for anti-SARs-CoV-2 antibodies). In contrast, N protein assays detect a protein present in live virus typically found in the respiratory tract. Therefore, samples for such testing may be in the form of a nasal swab that can be subsequently diluted in a lysing buffer. After dilution, the sample is clean enough that it does not require a plasma membrane for filtration. As a result, use of a single-inlet assay device (e.g., like assay device 700) was appropriate.

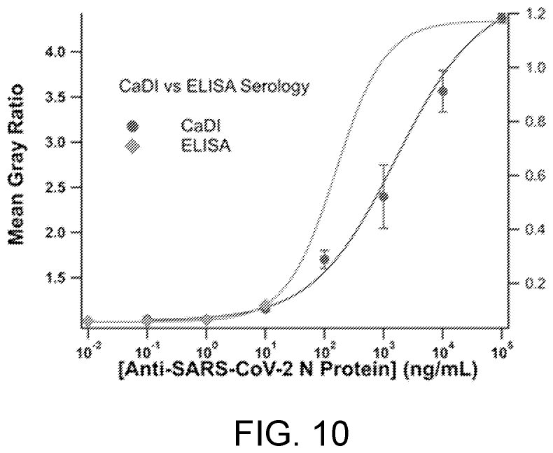

For the serology assay, a nitrocellulose membrane (e.g., test strip 610) was striped with SARs-CoV-2 nucleocapsid protein (NP). A first reagent pad (generally corresponding to dried enzyme label pad 614 of assay device 600) was prepared with dried anti-mouse-Horse radish peroxidase (HRP) while a second reagent pad (generally corresponding to dried substrate pad 616 of assay device 600) contained dried p-dimethylaminoazobenzene (DAB), a colorimetric substrate for HRP. The nitrocellulose membrane was blocked with StabilGuard and the buffer used in the buffer inlet contained 0.01% hydrogen peroxide to activate the HRP. FIG. 10 shows the dose-response curve obtained for the target anti-SARs-CoV-2-NP from whole blood compared to the same assay run with a traditional laboratory-based ELISA. As illustrated in FIG. 10, the results of the test validated the capability of the assay device to run an ELISA for identifying the presence of anti-SARs-CoV-2 antibodies. Notably and consistent with assay device devices described herein, the test required only two basic steps (providing a sample to a sample inlet and providing a buffer to a buffer inlet) with all subsequent routing and delivery to the nitrocellulose membrane resulting from natural capillary-driven flow through the microfluidic network of the assay device.

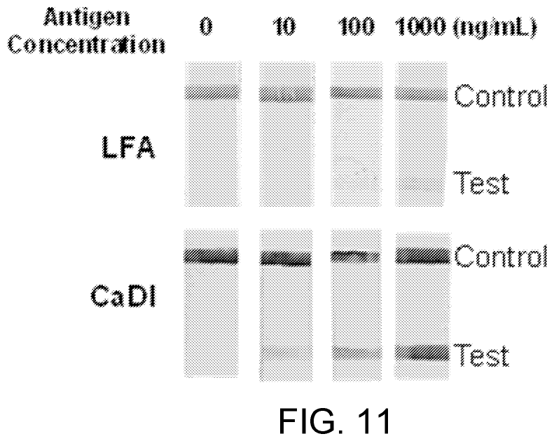

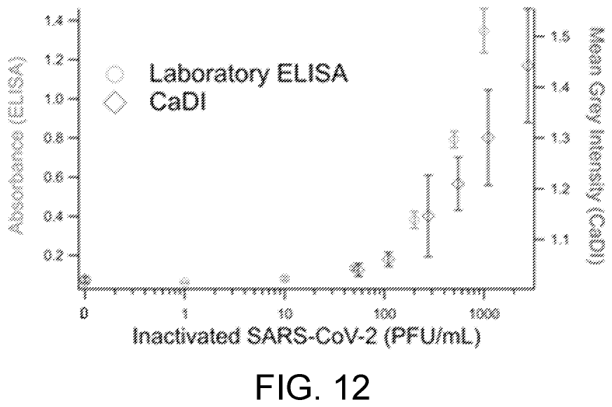

As noted above, the antigen assay was completed with a single-inlet device, such as assay device 700. The only changes that needed to be made to transition to the antigen assay were switches in the capture and detection antibodies so that SARs-CoV-2 N protein was the target. HRP was still used as the label, but tetramethylbenzidine (TMB) was used as the colorimetric substrate instead of DAB, providing more sensitive results. The antigen assay was compared to a laboratory ELISA and to a traditional lateral flow assay using gold nanoparticles as a label. The results of this comparison are illustrated in FIG. 11. The assay device performed similarly to the laboratory-based ELISA, as shown in FIG. 12. The assay device also outperformed the lateral flow assay by several orders of magnitude as shown in FIG. 11. As a result, the improvement in performance over the lateral flow assay validates that the assay device disclosed herein can outperform traditional point-of-care assays in terms of sensitivity without sacrificing ease-of-use.

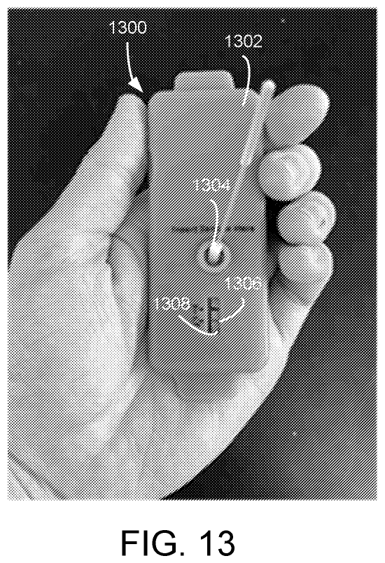

FIG. 13 illustrates an example assay device 1300 according to the present disclosure. As illustrated, assay device 1300 is a single inlet device including a housing 1302 within which a device body defining a microfluidic network is disposed (not shown). Housing 1302 further defines a first port 1304 for receiving a fluid (e.g., a combination of a sample and buffer). Housing 1302 further includes a second port 1306 for viewing a result of a test conducted using assay device 1300. More specifically, second port 1306 provides a view of a test strip 1308. As illustrated, test strip 1308 is configured to include each of a test line to display an outcome of a test and a control line for verifying proper function of assay device 1300.

As discussed herein, the device body and microfluidic network defined by the device body generally includes various pathways and dried reagents along the pathways to facilitate sequential delivery of rehydrated reagent to test strip 1308. Housing 1302 may be configured to be unsealed, thereby permitting venting of the microfluidic network as previously described.

Notably, assay device 1300 may be readily modified to accommodate multiple inlets and, as a result, to perform test that may require separate introduction of a sample and a buffer fluid. In such cases, assay device 1300 may be modified to have each of a sample inlet and a buffer inlet. The sample inlet may also include a filtration membrane or similar component for separating/processing the sample.

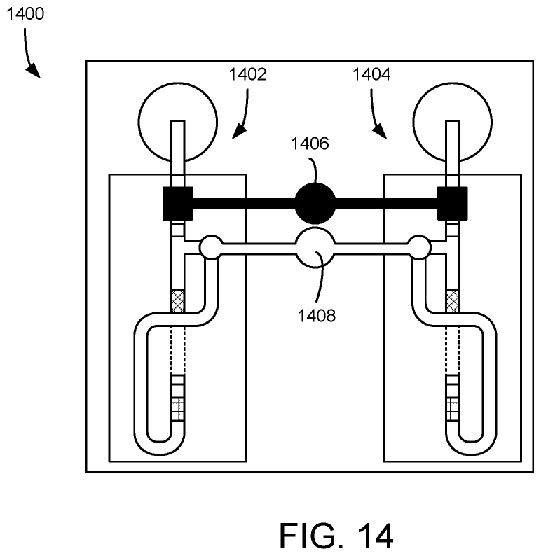

FIG. 14 illustrates a device 1400 including multiplexed assay device devices. The device 1400 includes a first assay device 1402 and a second assay device 1404 that share a common sample inlet 1406 and a common buffer fluid inlet 1408. Assay device 1402 and assay device 1404 may otherwise be substantially the same as any assay device disclosed herein. The device 1400 may be used to simultaneously perform multiple assays for a given sample. For example, in device 1400, first assay device 1402 is configured to perform a first assay while second assay device 1404 is configured to perform a second assay. Notably, except for shared common sample inlet 1406 and common buffer fluid inlet 1408, first assay device 1402 and second assay device 1404 may otherwise be isolated, thereby permitting use of reagents, etc. in one of the assay devices may be incompatible with reagents in the other assay device or that may otherwise interfere with results of the assay performed by one of the assay devices. Although illustrated as dual-inlet assay devices, the multiplexed concept illustrated in FIG. 14 may be readily adapted to single-inlet assay devices, multi-inlet assay devices, or any combination thereof.

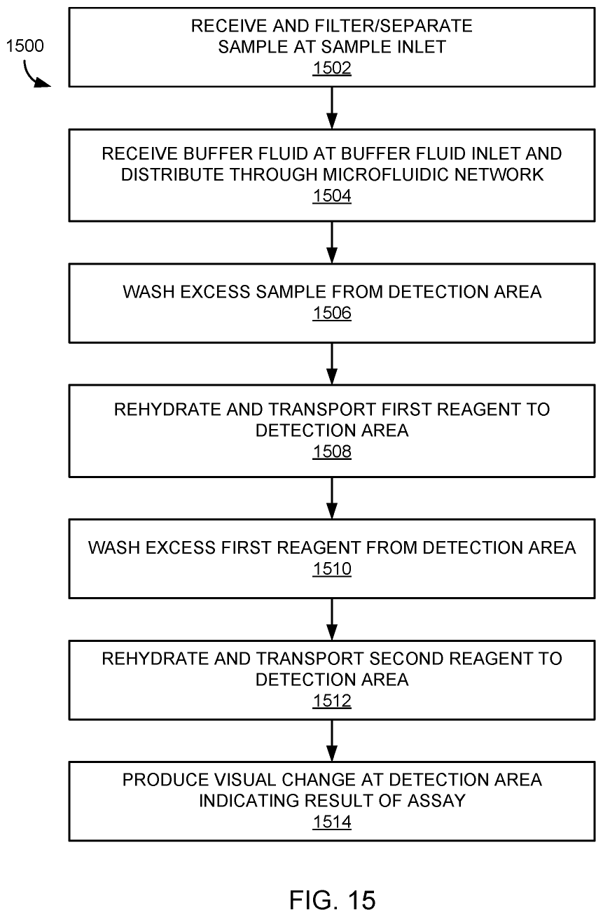

FIG. 15 is a flow chart of a first method 1500 of performing an assay using a multi-inlet assay device, such as assay device 600 of FIG. 6A-C. At operation 1502, a sample is received at a sample inlet of an assay device. The assay device includes a microfluidic network in communication with each of the fluid inlet and a colorimetric assembly including a detection area. In certain implementations, the sample is filtered, e.g., using a filtration membrane. The sample may also be driven, at least partially, to the detection area by capillary-driven flow when the sample is introduced.

At operation 1504, a buffer fluid is received at a buffer fluid inlet of the assay device. The buffer fluid may substantially fill the microfluidic network and addition of the buffer fluid may generally initiate capillary-driven flow through the microfluidic network. Capillary-driven flow may also be facilitated by a passive pump in communication with the microfluidic network.

At operation 1506, the buffer fluid flows across the detection area, washing excess sample from the detection area.

At operation 1508, a first rehydrated reagent is delivered to the detection area. More specifically, a first dried reagent, such as a dried enzyme label, may be disposed along a first path of the microfluidic network (e.g., in the form of a pad onto which the first reagent is dried). When the buffer fluid is added, the buffer fluid may rehydrate the first dried reagent and may initiate transportation of the first rehydrated reagent to the detection area.

At operation 1510, another portion of the buffer fluid may be transported across the detection area, washing excess first reagent from the detection area.

At operation 1512, a second rehydrated reagent is delivered to the detection area. More specifically, a second dried reagent, such as a dried substrate, may be disposed along a second path of the microfluidic network (e.g., in the form of a pad onto which the second reagent is dried). When the buffer fluid is added, the buffer fluid may rehydrate the second dried reagent and may initiate transportation of the rehydrated second reagent to the detection area.

At operation 1514, a result of the assay is visually indicated. For example, a stripe, pattern, or similar indicator may appear at the detection area in response to a product produced by delivery of the substrate.

Notably, devices in accordance with the present disclosure are configured to perform operations 1506-1514 of FIG. 15 automatically in response to receiving each of the sample and buffer fluid. Stated differently, the washes and reagent deliveries occur automatically due to capillary-driven flow and, as a result, do not require intervention by an end user beyond introduction of the sample and the buffer fluid. While operations 1508 (rehydration and delivery of first reagent) and 1512 (rehydration and delivery of second reagent) are generally discussed above and illustrated as sequential, rehydration of the first and second reagents and initial transportation of the first and second reagents may be simultaneous or overlap following introduction of the buffer fluid. However, the microfluidic network is configured such that the rehydrated second reagent arrives at the detection area after the first reagent and, in most applications, after a wash performed after delivery of the first reagent to the detection area.

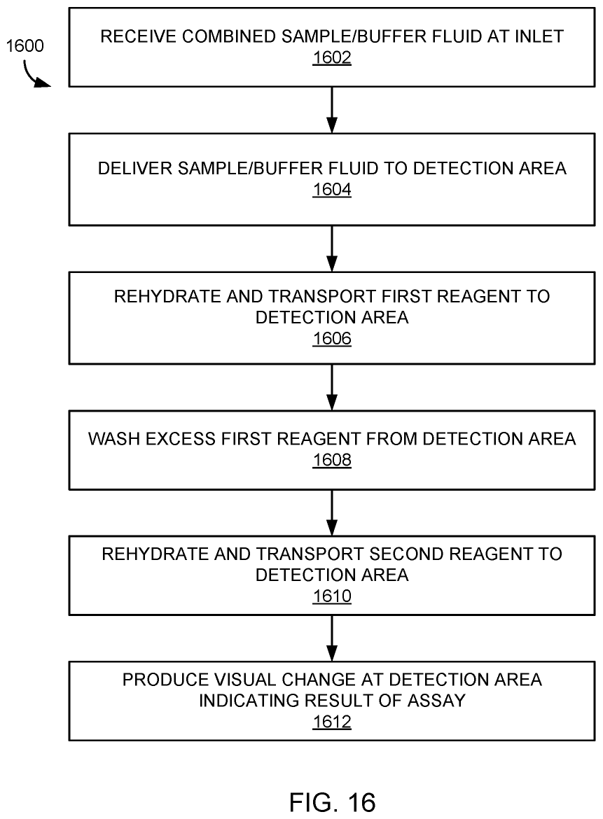

FIG. 16 is a flow chart of a second method 1600 of performing an assay using a single-inlet assay device, such as assay device 700 of FIG. 7. At operation 1602, a combined sample and buffer fluid is received at a fluid inlet of an assay device. The assay device includes a microfluidic network in communication with each of the fluid inlet and a colorimetric assembly including a detection area. The sample/buffer fluid may substantially fill the microfluidic network and addition of the sample/buffer fluid may generally initiate capillary-driven flow through the microfluidic network. Capillary-driven flow may also be facilitated by a passive pump in communication with the microfluidic network.

At operation 1604, the sample/buffer fluid flows across the detection area, delivering sample to the detection area.

At operation 1606, a first rehydrated reagent is delivered to the detection area. More specifically, a first dried reagent, such as a dried enzyme label, may be disposed along a first path of the microfluidic network (e.g., in the form of a pad onto which the first reagent is dried). When the sample/buffer fluid is added, the sample/buffer fluid may rehydrate the first dried reagent and may initiate transportation of the resulting rehydrated reagent to the detection area.

At operation 1608, another portion of the sample/buffer fluid may be transported across the detection area, washing excess first reagent from the detection area.

At operation 1610, a second rehydrated reagent is delivered to the detection area. More specifically, a second dried reagent, such as a dried substrate, may be disposed along a second path of the microfluidic network (e.g., in the form of a pad onto which the second reagent is dried). When the sample/buffer fluid is added, the sample/buffer fluid may rehydrate the second dried reagent and may initiate transportation of the rehydrated second reagent to the detection area.

At operation 1612, a result of the assay is visually indicated. For example, a stripe, pattern, or similar indicator may appear at the detection area in response to a product produced by delivery of the substrate.

Notably, devices in accordance with the present disclosure are configured to perform operations 1604-1612 of FIG. 16 automatically in response to receiving the sample/buffer fluid. Stated differently, the washes and reagent deliveries occur automatically due to capillary-driven flow and, as a result, do not require intervention by an end user beyond initial introduction of the sample/buffer fluid. While operation 1606 (rehydration and delivery of first reagent) and operation 1610 (rehydration and delivery of second reagent) are generally discussed above and illustrated as sequential, rehydration of the first and second reagents and initial transportation of the first and second reagents may be simultaneous or overlap following introduction of the sample/buffer fluid. However, the microfluidic network is configured such that the rehydrated second reagent arrives at the detection area after the first reagent and, in most applications, after a wash performed after delivery of the first reagent to the detection area.

Considering the foregoing, the assay device disclosed herein represents a substantial innovation in disposable assays. Like other at-home assays, it is easy to operate, and results can be clearly interpreted. However, the analytical performance is enhanced by its ability to sequentially and automatically add and wash reagents from a nitrocellulose test zone. This capability allows the device to function as a disposable ELISA, opening new applications for sensitive and selective at-home detection of biomolecules at low concentrations.

Various modifications and additions can be made to the exemplary implementations discussed without departing from the scope of the present invention. For example, while the implementations described above refer to specific features, the scope of this invention also includes implementations having different combinations of features and implementations that do not include all the described features. Accordingly, the scope of the present invention is intended to embrace all such alternatives, modifications, and variations together with all equivalents thereof.

1. An assay device comprising:
a colorimetric testing assembly including a detection area;
a fluid inlet;
a microfluidic network including a first path extending to the detection area and a second path extending to the detection area;
a first dried reagent disposed along the first path; and
a second dried reagent disposed along the second path,
wherein, when a fluid is provided to the fluid inlet, a first portion of the fluid rehydrates the first dried reagent to produce a first rehydrated reagent, a second portion of the fluid rehydrates the second dried reagent to produce a second rehydrated reagent, and the first rehydrated reagent and the second rehydrated reagent are sequentially delivered to the detection area by capillary-driven flow.

2. The assay device of claim 1, further comprising a pad, wherein the pad is disposed within the first path and contains the first dried reagent or wherein the pad is disposed within the second path and contains the second dried reagent.

3. The assay device of claim 1, wherein the first path includes a first surface on which the first dried reagent is disposed or the second path includes a second surface on which the second dried reagent is disposed.

4. The assay device of claim 1, wherein the first rehydrated reagent arrives at the detection area before the first rehydrated reagent.

5. The assay device of claim 1, further comprising a sample inlet, wherein the sample inlet is separate from the fluid inlet and in communication with the microfluidic network.

6. The assay device of claim 1, further comprising a sample inlet, wherein the sample inlet is separate from the fluid inlet and in communication with the microfluidic network, and wherein the sample inlet includes a filtration membrane.

7. The assay device of claim 1, wherein the first path is shorter than the second path.

8. The assay device of claim 1, wherein the microfluidic network is configured such that the fluid arrives at the detection area by the first path before the fluid arrives at the detection area by the second path.

9. The assay device of claim 1, further comprising a body formed from alternating layers of plastic and double-sided adhesive, wherein the microfluidic network is formed by the layers of film and double-sided adhesive.

10. The assay device of claim 1, further comprising a vent in communication with the microfluidic network.

11. The assay device of claim 1, further comprising:
a second colorimetric testing assembly including a second detection area; and
a second microfluidic network in communication with each of the fluid inlet and the second detection area.

12-18. (canceled)

19. An assay device comprising:
a colorimetric testing assembly including a detection area;
a microfluidic network in communication with the colorimetric testing assembly, wherein the microfluidic network is defined within a device body formed from alternating layers of film and double-sided adhesive;
a fluid inlet in communication with the microfluidic network;
a first path extending to the detection area;
a dried enzyme label disposed along the first path;
a second path extending to the detection area, the second path being longer than the first path; and
a dried substrate disposed along the second path.

20. The assay device of claim 19, wherein the fluid inlet is a buffer fluid inlet, the assay device further comprising a sample inlet in communication with the microfluidic network.

21. An assay device comprising:
a colorimetric testing assembly including a detection area wherein, when a fluid is provided to a fluid inlet, a first portion of the fluid rehydrates a first dried reagent to produce a first rehydrated reagent, a second portion of the fluid rehydrates a second dried reagent to produce a second rehydrated reagent, and the first rehydrated reagent and the second rehydrated reagent are sequentially delivered to the detection area by capillary-driven flow.

22. The assay device of claim 21, further comprising
a microfluidic network in communication with the colorimetric testing assembly, wherein the microfluidic network is defined within a device body formed from alternating layers of film and double-sided adhesive;
wherein the fluid inlet is in communication with the microfluidic network;
a first path extending to the detection area;
a dried enzyme label disposed along the first path;
a second path extending to the detection area, the second path being longer than the first path; and
a dried substrate disposed along the second path.

23. The assay device of claim 21, further comprising a pad, wherein the pad is disposed within a first path and contains a first dried reagent or wherein the pad is disposed within a second path and contains a second dried reagent.

24. The assay device of claim 22, wherein the first path includes a first surface on which the first dried reagent is disposed or the second path includes a second surface on which the second dried reagent is disposed.

25. The assay device of claim 21, wherein the first rehydrated reagent arrives at the detection area before the first rehydrated reagent.

26. The assay device of claim 21, further comprising a sample inlet, wherein the sample inlet is separate from the fluid inlet and in communication with a microfluidic network.

27. The assay device of claim 22, wherein the fluid arrives at the detection area by the first path before the fluid arrives at the detection area by the second path.
